(19) United States
(12) Patent Application Publication
(10) Pub. No.: US 2006/0177497 A1
(43) Pub. Date: Aug. 10, 2006

(54) Gellan gum based oral controlled release dosage forms-a novel platform technology for gastric retention

(75) Inventors: David Hoikhman, Hadera (IL); Yoram Sela, Raanana (IL)

Correspondence Address: BROWDY AND NEIMARK, P.L.L.C.;624 NINTH STREET, NW, SUITE 300, WASHINGTON, DC, 20001-5303, US

(73) Assignee: BIO DAR LTD., Yavne (IL)

(21) Appl. No.: 10/565,593

(22) PCT Filed:
(86) PCT No.: PCT/IL04/00654

Related U.S. Application Data
(60) Provisional application No. 60/488,421, filed on Jul. 21, 2003.

(51) Int. Cl. A61K 9/48; A61K 9/26

(52) U.S. Cl. 424/451; 424/469

(57) ABSTRACT

A controlled-release dosage form is described, which comprises a matrix formed of ingredients (a) and (b): (a) gellan gum, and (b) one or more hydrophilic polymers; and further comprising a drug incorporated within said matrix. The invention also describes a method for the preparation of said controlled-release dosage forms.

### BACKGROUND

Orally administrated dosage forms are is most cases, the preferred way of medication. However, numerous drugs administrated per-os are absorbed efficiently only in the upper gastrointestinal tract, namely, the stomach and the proximal section of the small intestine. The passage of drugs from the stomach to the intestine is normally too fast (usually, between one or two hours), strongly limiting their bioavailability. Since the residence time of drug at the site of optimal absorption largely determines its bioavailability, it is apparent what prolonging the retention of the drug-containing device in the proximal gastrointestinal tract is of the utmost importance. Delivery of a drug at a constant rate from the gastric device could assist in maintaining constant level of the released drug and overcome the blood and tissue variable concentration due to diurnal variation in the intake of the drug by the patients. Long-term gastric retention device could ease medical treatment and improve patient's compliance.

A gastric-retentive device for long-term drug release can significantly improve treatments with drugs that are taken for long periods, as in the case of chronic diseases, hormonal treatments, as well as simplify treatments, as well as simplify treatments that combine several different drugs.

Various approaches to achieve gastric retention of controlled release dosage forms were developed over the years. However, in spite of the diversity of approaches a limited number of devices actually reach the clinics, and those meet only limited success and fail to attain residence time longer then 24 hours.

The controlled delivery of drugs has witnessed remarkable progress during the last decade. Nevertheless, orally administrated dosage forms still encounter substantial obstacles and remain a major challenge. One of the main difficulties faced by controlled delivery systems administered per-os, is to attain optimal plasma drug levels in a reproducible and predictable manner.

The principal motor tasks of the stomach are to liquefy the meal (digestive function) and to deliver it into the intestine at a rate that matches the processing capability of the intestine (reservoir function).

It is widely accepted that the stomach can be divided into two main regions, depending on the function performed: 1) the proximal stomach—mainly the fundus and the upper gastric body—behaves as a depot, by modulating the tonic of its muscular walls, and accommodating its content. 2) the distal stomach (antrum), which, in contrast to the proximal stomach, generates peristaltic phasic contractions that grind solid particles. The solid bolus is ground until the particle size is small enough (<2.0 mm) to permit passage into the duodenum.

The motor activity of the distal stomach is characterized by peristaltic waves originated from the mild-stomach to the duodenum. The electrical pacing of this activity is located in the muscular wall of the proximal gastric body. The pacemaker discharges at a frequency of 3 cycles per minute, and spreads circumferentially and distally. In the presence of food or other distending sources, it converts to action potentials and muscle contractions. The peristaltic wave generated is lumen-obliterating in the distal 2 cm of the antrum. Solid food is retained there for further grinding.

An additional motor form, termed the Migratory Motor Complex (MMC), is responsible for the emptying of indigestible solids—usually in excess of 5 mm—which cannot be emptied with digestible solids. The MMC are powerful “housekeeping” waves that are inhibited by feeding, are stimulated by fasting, and occur every 60-120 minutes.

The transit of a dosage form though the gastrointestinal tract is largely affected by physiological factors, especially by the presence or absence of food in the stomach, as well as by the chemical and physical properties of the dosage form, such as its hydrophilicity, its size and stiffness, and also by mucosal receptors in the small intestine that are sensitive to caloric, osmolar and acid loads. Depending on these factors, the emptying process can range from several minutes up to several hours and represents, therefore, the primary limit step.

It is accepted almost consensually, that only solid particles smaller than 2 mm are able to pass the pylorus. This is mainly due to the fact that the pyloric sphincter closes, as the peristaltic wave approaches the terminal antrum, and therefore, larger particles will remain in the stomach until they are further reduced in size. It is the combined mechanical effect of this grinding process and the acid-peptic digestive attack that reduces solid food into chymouslike substance, able to outflow into the small intestine. While there is no consensus about the size dependence of gastric emptying by the MMC, the data is the literature suggest that, for oral dosage forms to remain in the stomach in the fasted state, their size has to be larger than 15 mm. The difficulties to develop devices in that size range is further enhanced, due to the variability in their response time.

The objective of gastric-retentive devices is to deliver drugs intra-gastrically, in a controlled manner, over relatively long time periods. The medication to be considered must fit the following criteria:
1. Large therapeutic range: deviations from the amount of released drug, above or below the predicted level, will not cause any significant symptoms.
2. Safety: Over-dose will not endanger the treated subjects.

Many groups of medications comply with these requirements and are potential candidates for delivery by the proposed device. Among them: Analgesics, Anxiolytics, Antimigroine drugs, Sedatives, Antipsihotics, Anticonvulsants, Antiparcinsons, Antiallergic drugs, Antidepressants, Antiemetics, Astma-profilactics, Gastric-hypoacidics, Anticonstipation drugs, Intestinal antiinflammatory agents, Antihelmintics, Antianginals, Diuretics, Hypolipidemic agents, Anti-inflammatory drugs, Hormones, Vitamins, Antibiotics.

There are several common approaches to increase gastric retention:

a) Intragastric Floating Systems

These devices are based upon floating in the gastric fluid.

Three major techniques are used to generate buoyancy in the gastric fluid:
1. Gas containing floating systems usually generates CO₂ by mixture of bicarbonate and gastric fluid (or another acid incorporated into the device). The gas is trapped in the system, causing it to float, prolonging its residence in the stomach.
2. Low-density core systems are made of buoyant materials that do not have to undergo any chemical or physical change, to ensure their buoyancy. Around the low-density core, which contains air, gels or other materials, there is an outer layer that releases the drug in a controlled manner.
   3. Hydrodynamic balanced systems contain mainly a gel forming hydrophilic polymer, which, upon contact with the gastric fluid, from a gelatinous shell, which releases the drug. Its buoyancy is ensured by its dry or hydrophobic core.

The main disadvantage of floating systems stems from their short intragastric residence time (usually less then few hours). These systems do exhibit, some improvement in the absorption of various agents in the upper GI tract, but do not achieve longer gastric retention. In addition, their action is dependent on the amount of food and water in the stomach, which may cause non uniform performance of these systems.

b) High-Density Systems

High-density devices are based on the sinking of the device to the bottom of the stomach, and are usually made of steel or other heavy materials. Initially, this approach looked promising, but many studies have shown no appreciable gastric retention.

The main drawbacks of this technique are its dependence on the position of the stomach and the need for larger and heavier systems for obtaining the desired retention.

A combination of this approach with swellable system was suggested to enlarge its size while keeping its high density.

c) Mucoadhesive Systems

The bioadhesive systems are based on their ability to stick to the mucous layer in the stomach. Due to their adhesiveness to the gastric mucosa, they were expected to remain in the stomach, during the mucous layer turnover.

Nevertheless, the results were disappointing, and no substantial prolongation of the residence time in the stomach has been achieved.

The main problem of the mucoadhesive devices is their tendency to bind almost to any other material they come in contact with—i.e. gelatin capsules, proteins and free mucous—in the gastric fluid. Another major obstacle is the pH-dependent bio-adhesiveness of some of these materials. Higher than normal gastric pH levels, reduce dramatically the adhesion strength of these systems, and therefore their effectivity

Substantial progress (particularly, in ensuring specificity of the mucoadhesive material to the gastric wall) has to be made before these systems become viable.

d) Magnetic Systems

Small magnet-containing tablets attached to a drug releasing system, are prevented from leaving the stomach, by an extra-corporeal magnet, placed over the stomach. Even through various studies reported some success, the viability of these system is in doubt, because of the need to carry an extra-corporeal magnet and to place it very accurately, in order to obtain the desired results. New, more convenient ways to apply a magnetic field have to be found to improve this concept.

e) Unfoldable/Extendable/Expandable Systems

Expandable systems are based on a sharp dimensional change, following arrival to the stomach.

Several Methods were Proposed:
1. Hydrogels that swell upon their contact with gastric fluid.
2. Osmotic devises that contain salts or sugars, surrounded by a semi permeable membrane.
3. Systems containing a low boiling liquid, that turns into gas at body temperature and inflates the device to its desired size, while, simultaneously to the swelling of the system, a period of sustained release begins.

There are several problems regarding these systems, including the slow swelling rate of some of them (up to several hours) failing, therefore, to retain the device intra-gastrically.

In addition, the ability to swell to the desired size and the degradation process still pose a substantial challenge to the feasibility to the swelling systems. Superporous hydrogels have dealt with some of these problems with some degree of success, and are discussed later. The low temperature boiling gas systems are very sensitive to temperature fluctuations, resulting in determinant events such as premature opening in the esophagus.

Unfoldable and extendible systems are based on a mechanical device which unfolds or extends from its initially small size, to an extended form that prevents its passing through the gastric pylorus. The active agent may be a part of the polymer composing the retentive system or, alternatively, attached to it as a different component, or laminated over or inside it.

While experiments conducted on beagle dogs were rather encouraging, a much faster passage was observed in humans, indicating the need for optimization of these devices. Another problem of these systems is their storage in their folded form, which tends to reduce their elasticity and limits their rapid unfolding once in the stomach. The manufacturing of these devices often poses an additional challenge, due to the multi-component nature of these devices, their complex form and the need to fold and hold it in its folded form.

f) Superporous Biodegadable Hydrogel Systems.

This approach is based upon swelling of unique hydrogel systems, Superporous hydrogels were synthesized by crosslinking polymerization of various vinyl monomers in the presence of gas bubbles formed by chemical reaction of acid and NaHCO₂. The difference between these devices and those described earlier, is the much higher swelling levels attained by system comprising. Another advantage of superporous hydrogels is their ability to swell much faster than the conventional hydrogels (minutes as opposed to hours, respectively). Their major disadvantage pertains to their weak mechanical properties and the resulting short residence times attainable by these systems. Even when reinforcing agents are added, these devices remain weak and do not perform satisfactorily. Clearly, therefore, much progress has to be made, before these systems become clinically feasible.

g) Matrix Systems

Matrix systems can be subdivided into different categories, these being dispersed and porous systems where the matrix-forming material does not undergo dimensional changes in contact with the gastric fluid. The advantage of non-erodible dispersed matrix systems over reservoir and erodible systems is that they are relatively insensitive to changes in mixing and stirring conditions because diffusion is the rate-controlling factor. Conventional dispersed systems suffer from non-linear concentration-time release, due to the longer distance that the drug in deeper layers of the matrix must travel to exit the delivery system. During both drug dissolution and diffusional process, the boundary layer moves back into the matrix while its surface area is maintained.

To overcome this problem of non-linear release and to facilitate zero order drug delivery, studies have been performed on disperse matrices that contain increasing concentrations of drug as the core is penetrated and have been shown to alleviate the problem of non-linear release.

Drug release from such systems is based upon the fact that the dissolution medium surrounding the matrix device initially dissolves and leaches out drug from the surfaces of the device, but at this process continues with time, the dissolution medium travels further into the matrix and the drug then has to dissolve into the medium and then leave via diffusion along the porous water filled paths, created by the gradual ingress of the dissolution medium. Hence, before the tablet is placed in the dissolution medium, there are relatively few porous paths within matrix. Drug release rates would therefore be expected to change with drug solubility and drug loading.

Hydrophylic Matrices

Hydrophilic systems usually consist of a significant amount of drug dispersed in and compressed together with a hydrophylic hydrogel forming polymer and may be prepared together with either a soluble or insoluble filler. When these systems are placed in the dissolution medium, Dissolution occur by a process that is a composite of two phenomena: in the early stages of dissolution, polymer (and) drug dissolution begins, the polymer dissolving due to chain disentanglement or hydrogel formation as a result of cross-linking. The rate constant for drug release from a swellable matrix is a function of the diffusion coefficient of the drug matrix, which depends on the free volume of water.

In view of the foregoing there is a long felt need for a gastric retention system for pharmaceuticals which overcomes the disadvantages of the prior art.

Gellan gum, first discovered in 1978, is produced by the microorganism Pseudomonas elodea. The constituent sugars of gellan gum are glucose, glucoronic acid and ramnose in the molar ratio of 2:1:1. These are linked together, as shown in FIG. 1, to give a primary structure comprising of a linear tetrasacharide repeating unit. In gellan gum's common form (also referred to as the high acyl form) two low acyl substituents, acetate and glycerate, are present. Both constituents are located on the same glucose residue, and on average, there is one glycerate per repeating unit and one acetate per every two repeating unit. In low acyl gellan gum, the acyl groups are removed completely.

Light scattering and intrinsic viscosity measurements give a molecular mass of approximately 5×10⁵ Daltons for the deacylated gum. X-ray diffraction analysis of oriented fibers shows that gellan gum exists as a three-fold, left-handed, parallel double helix. The pair of molecules that constitute the helix is stabilized by hydrogen bonds at each carboxylate group. In the potassium salt (FIG. 2) of the deacylated material, the potassium ion is coordinated to the carboxylate group, which in turn is involved in interchain hydrogen bonds. The potassium ions are located on the outside of the helix and, besides providing helix stabilization, they allow the helix to aggregate. In the calcium salt form, the model is similar except the divalent calcium replaces two potassium ions and one molecule of water. In these salt forms of the gel, helix aggregation is responsible for the gel's brittle character.

Gellan gum functions as a structuring and gelling agent in a wide variety of foods, water based dessert gels etc. In pharmaceutical applications the Gellan use is limited to tablets coating and disintegration purposes.

### DESCRIPTION OF THE INVENTION

The following description is illustrative of preferred embodiments of the invention. The following description is not to be construed as limiting, it being understood that the skilled person may carry out many obvious variations to the invention.

It has surprisingly been found that Gellan gum has the ability to form fast swellable gels when combined with other hydrophilic polymers and to form strong gels when adding the Gellan gum and hydrophilic polymer combination to the gastric environment. Superior synergistic effects between the Gellan gum and the polymers were found when the hydrophilic polymers had homopolysaccharide backbone. Non-limiting examples of hydrophilic polymers are: guar gum, heteropolysaccharides, Carmelose, hydroxypropylmethylcellulose (BPMC), carboxymethylcellulose sodium, and Xantan gum.

A unique gastro retentive platform technology of the present invention is based on these findings, introducing a controlled-release dosage form comprising a matrix and at least one active drug, whereas the matrix comprises Gellan gum, one or more hydrophilic polymers, and optionally further comprising other non-active pharmaceutically acceptable additives, such as metal ions, colorants, taste maskers, dietary components, excipients, binding agents, coatings, preservatives etc., and mixtures thereof.

Combining homo and heteropolysaccharides was found to produce faster gelation of the systems, by physical cross-linking of the polymer chains. The “combined” gel is characterized by its fast forming and rigidity characteristics. Therefore a preferred embodiment of the invention is a dosage form, whereas the matrix comprises Gellan gum, a homopolysaccharide polymer and a heteropolysaccharide polymer, and optionally other pharmaceutically acceptable non-active additives.

The present invention provides synergistically interacting controlled release dosage form systems based on gellan gum combinations.

Yet another embodiment of the invention is dosage form in an orally-administered form.

Said orally-administered dosage forms can be in a variety of forms such as fine granules, granules, pills, tablets and capsules. Preferred dosage forms are tablets.

According to yet a further aspect of the invention, the controlled release dosage form systems of the present invention are prepared in the following manner:
1. Homogenizing the matrix components with the active drug via mechanical means, resulting in a premix.
2. Adding to the premix a combination of water and one or more hydrophilic solvents, obtaining a pharmaceutically acceptable wet granule. The addition of the hydrophilic solvents prevents premature gelation or swelling during the manufacturing process.
3. Drying the wet granulate via conventional drying methods, obtaining a dried granulate, to enable easy screening in the next step.
4. Screening the dried granulate through a sieving system to obtain a screened granulate of a size suitable for post-processing preferably in the range of 0.3 to 1 mm.
5. Adding a lubricant to the screened granulate, whereas the lubricant is any of a large variety of pharmaceutically acceptable gelling lubricants, provided that the lubricant is not a multi-valent salt. Mixing time varies on the lubricant and batch size.

The present invention is advantageous in that it provides dosage forms with improved gel stability and which are easily formed in vivo, directly in the gastric environment.

Furthermore, the dosage forms are advantageous for providing gels of a particle size which prevents the dosage forms from exiting the stomach (also referred to as the upper part of the gastric intestinal (GI) system), thus prolonging the release of the drug and increasing the drug bioavailability and efficiency.

The drug suitable for application the present dosage form is selected from the group comprising of anti-inflammatory drugs, antiepileptics, hypnotic sedatives, antipyretic analgesics, stimulants, antihypnotics, drugs for vertigo, drugs for the central nervous system, skeletal muscle relaxants, drugs for the autonomic nervous system, autonomic ganglionic blockers, drugs for the peripheral nervous system, opthalmic drugs, drugs for sense-organs, cardiacs, antiarrhythmics, diuretics, antihypertensives, vasoreinforcements, vasoconstrictors, vasodilators, antiarteriosclerotics, circulatory drugs, respiratory stimulants, antitussive expectorants, drugs for respiratory organs, peptic ulcer drugs, stomachic digestants, antacids, cathartics, cholagogues, digestive drugs, hormonal agents, urinary tract disinfectants, uterotonics, urogenital drugs, drugs for anus diseases, vitamins, nutritive roborants, drugs for blood or body fluid, drugs for hepatic diseases, antidotes, habitual intoxication drugs, antipodagrics, enzyme preparations, antidiabetics, cell activation drugs, antitumor agents, antibiotics, chemotherapeutic agents, and arthritis therapeutics.

In another embodiment of the invention, the drug employed in the dosage form has preferred absorption at the upper parts of the gastric system.

More preferably, the drug employed in the dosage form is selected from: clarithromycin, metformin, azidotimidine, orlistat, ciprofloxacin and levodopa.

### BRIEF DESCRIPTION OF THE DRAWINGS

FIG. 1—Schematic representation of the chemical repeating-unit. [A, B, C and D are β-D glucose, β-D-glucuronate, β-D-glucose, and α-L-ramnose respectively]

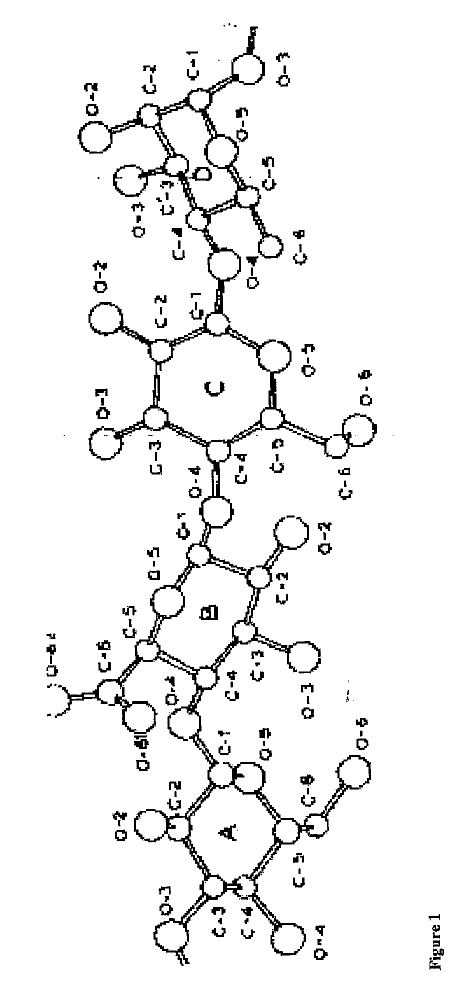

FIG. 2—Side view of the double helix in stereo showing the OH—O hydrogen bonds within the molecule

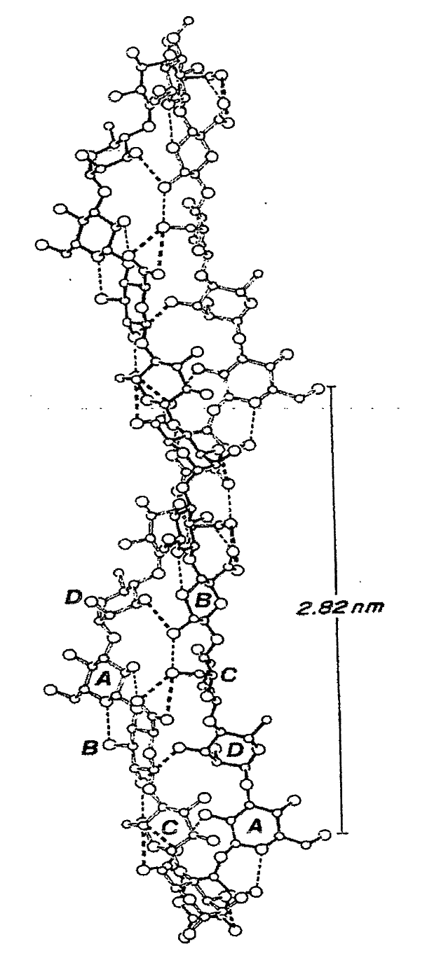

### EXAMPLES

### Example 1

### Sample Preparation

All samples were prepared according to the following procedure:

Metformin was used as the drug model in all of the samples. All compositions further contain between 20 to 80 ml ethanol:water mixtures for every 150 gr. of dry components.
1. The drug was premixed for 2 minutes using a Diosna type high shear granulator.
2. The premix was then mixed for 2 minutes with ethanol to produce a wet granulate.
3. The wet granulate was dried for 30 minutes using a Uniglatt, at an inlet air temp. of 50° C., and an outlet inlet air temp. of 46° C.
4. The composition was then screened through a 0.6 mm sieve.
5. The screened composition was lubricated for 10 minutes with polyethyleneglycol (PEG 6000) and then compressed into oval shaped tablets using a Riva rotary type D tabletting machine.

### Example 2

### Dosage Form Composition

Tablets were prepared according to the procedure of Example 1, whereas the dry ingredients of the matrix were in the following quantities:

| | | |
|---|---|---|
| Metformin HCl | 10 | g |
| Gellan gum low-acyl | 45 | g |
| Guar gum | 45 | g |
| CaCl₂ × 2H₂O | 0.08 | g |

The resulting tablets produce, after wetting, a dense and stable gel for more than 24 hrs in Gastric Fluid Simulation (GFS).

### Example 3

### Dosage Form Composition

Tablets were prepared according to the procedure of Example 1, whereas the dry ingredients of the matrix were in the following quantities:

| | | |
|---|---|---|
| Metformin HCl | 10 | g |
| Gellan gum low-acyl | 25 | g |
| Guar gum | 25 | g |
| HPMC (grade: 4KM premium) | 40 | g |
| PEG 6000 | 0.39 | g |

The resulting tablets produce, after wetting, a dense and stable gel for more than 24 hrs in GFS.

### Example 4

### Dosage Form Composition

Tablets were prepared according to the procedure of Example 1, whereas the dry ingredients of the matrix were in the following quantities:

| | | |
|---|---|---|
| Metformin HCL | 10 | g |
| Gellan gum low-acyl | 45 | g |
| Carboxymethylcellulose sodium | 45 | g |
| HPMC (grade: K100M premium) | 0.3 | g |

The resulting tablets produce, after wetting, a dense and stable gel for more than 5 hrs in GFS.

### Example 5

### Dosage Form Composition

Tablets were prepared according to the procedure of Example 1, whereas the dry ingredients of the matrix were in the following quantities:

| | | |
|---|---|---|
| Metformin HCL | 10 | g |
| Gellan gum low-acyl | 30 | g |
| Guar gum | 30 | g |
| Carboxymathylcellulose sodium | 30 | g |
| HPMC (grade: K100M premium) | 0.39 | g |

The resulting tablets produce, after wetting, a dense and stable gel for more than 1 week in GFS.

### Example 6

### Dosage Form Composition

Tablets were prepared according to the procedure of Example 1, whereas the dry ingredients of the matrix were in the following quantities:

| | | |
|---|---|---|
| Metformin HCL | 10 | g |
| Gellan gum low-acyl | 45 | g |
| Xanthan gum | 45 | g |
| HPMC (grade: K100M premium) | 0.37 | g |

The resulting tablets produce, after wetting, a dense and stable gel for more than 24 hrs in GFS.

### Example 7

### Dosage Form Composition

| | | |
|---|---|---|
| Metformin HCL | 11 | g |
| Gellan gum high-acyl | 4.5 | g |
| Carboxymethylcellulose sodium | 4.5 | g |
| Guar gum | 1 | g |

The resulting tablets produce, after wetting, a dense and stable gel for more than 24 hrs in GFS.

While embodiments of the invention have been described by way of illustration, it will be apparent that the invention may be carried out with many modifications, variations and adaptations, without departing from its spirit or exceeding the scope of the claims.

1. A controlled-release dosage form comprising a matrix formed of the following ingredients (a) and (b):
(a) gellan gum, and
(b) one or more hydrophilic polymers;
and further comprising at least one drug incorporated within said matrix;

2. The dosage form according to claim 1, wherein said ingredient (b) is selected from the group comprising: guar gum, hydroxypropyl methylcellulose, carboxymethyl cellulose sodium salt, xantan gum.

3. Dosage forms according to claim 1 comprising a combination of guar gum and carboxymethyl cellulose as component (b).

4. Dosage forms according to claim 1 comprising HPMC as component (b).

5. The dosage form according to claim 1, wherein at least one drug is selected from the group comprising of anti-inflammatory drugs, antiepileptics, hypnotic sedatives, antipyretic analgesics, stimulants, antihypnotics, drugs for vertigo, drugs for the central nervous system, skeletal muscle relaxants, drugs for the autonomic nervous system, autonomic ganglionic blockers, drugs for the peripheral nervous system, opthalmic drugs, drugs for sense-organs, cardiacs, antiarrhythmics, diuretics, antihypertensives, vasoreinforcements, vasoconstrictors, vasodilators, antiarteriosclerotics, circulatory drugs, respiratory stimulants, antitussive expectorants, drugs for respiratory organs, peptic ulcer drugs, stomachic digestants, antacids, cathartics, cholagogues, digestive drugs, hormonal agents, urinary tract disinfectants, uterotonics, urogenital drugs, drugs for anus diseases, vitamins, nutritive roborants, drugs for blood or body fluid, drugs for hepatic diseases, antidotes, habitual intoxication drugs, antipodagrics, enzyme preparations, antidiabetics, cell activation drugs, antitumor agents, antibiotics, chemotherapeutic agents, and arthritis therapeutics.

6. The dosage form according to claim 5, wherein the drug has preferred absorption at the upper parts of the gastric-intestine.

7. The dosage form according to claim 6, wherein the drug is selected from: clarithromycin, metformin, azidotimidine, orlistat, ciprofloxacin, levodopa.

8. The dosage form according to claim 1, wherein the dosage form further comprises other non-active pharmaceutically acceptable additives, such as metal ions, colorants, taste maskers, dietary components, excipients, binding agents, coatings, preservatives and mixtures thereof.

9. The dosage form according to claim 1, in an orally-administered form.

10. The oral dosage form according to claim 8, further processed in the form of tablets, caplets, vegecaps, and capsules.

11. A method for the preparation of controlled-release dosage forms, comprising the following steps:
(a) Homogenizing the matrix components with the active drug via mechanical means, resulting in a premix.
(b) Adding to the premix a combination of water and one or more hydrophilic solvents, obtaining a pharmaceutically acceptable wet granule.
(c) Drying the wet granulate via conventional drying methods, obtaining a dried granulate.
(d) Screening the dried granulate through a sieving system to obtain a screened granulate of a size suitable for post-processing.
(e) Adding a lubricant to the screened granulate
